(19) United States
(12) Patent Application Publication
(10) Pub. No.: US 2008/0275241 A1
(43) Pub. Date: Nov. 6, 2008

(54) Polymorphic Forms of Dolasetron Base and Processes of Preparing Dolasetron Base, Its Polymorphic Forms and Salt Thereof

(76) Inventors: Venkatasubramanian Radhakrishnan Tarur, Maharashtra (IN); Dhananjay Govind Sathe, Maharashtra (IN); Nandu Baban Bhise, Maharashtra (IN); Kamlesh Digambar Sawant, Maharashtra (IN); Tushar Anil Naik, Maharashtra (IN); Neeraj Srivastav, Maharashtra (IN); Raviraj Bhatu Deore, Maharashtra (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 12/158,708

(22) PCT Filed: Dec. 22, 2006
(86) PCT No.: PCT/IN06/00500

§ 371 Date: Jul. 16, 2008

(30) Foreign Application Priority Data
Dec. 23, 2005 (IN) 1610/MUM/2005
Dec. 29, 2005 (IN) 1635/MUM/2005

(51) Int. Cl. C07D 455/03

(52) U.S. Cl. 546/94

(57) ABSTRACT

The present disclosure relates to a process for the preparation of endo-hexahydro-8-(3-indolylcarbonyloxy)-2,6-methano-2H-quinolizin-3(4H)-one or Dolasetron base. It also discloses a process for the preparation of endo-hexahydro-8-(3-indolylcarbonyloxy)-2,6-methano-2H-quinolizin-3(4H)-one mesylate or Dolasetron mesylate. Further, the present disclosure relates to a process for producing Form I of Dolasetron base, and to the novel crystalline polymorphs, Form II, III, IV and V of Dolasetron base and industrial processes for producing them.

This specification claims priority from 1610/MUM/2005 dt 23/12/2005 and 1635/MUM/2005 dt 29.12.2005

### TECHNICAL FIELD

The present disclosure relates to a process for the preparation of endo-hexahydro-8-(3-indolylcarbonyloxy)-2,6-methano-2H-quinolizin-3(4H)-one or Dolasetron base of structural formula (A) and a process for the preparation of endo-hexahydro-8-(3-indolylcarbonyloxy)-2,6-methano-2H-quinolizin-3(4H)-one mesylate or Dolasetron mesylate of structural formula (1).

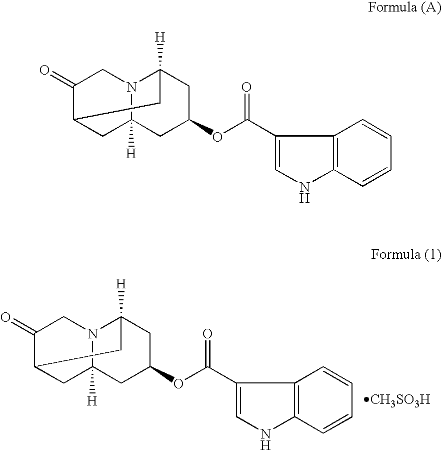

Further, it relates to novel crystalline polymorphs of Dolasetron base of structural formula (A) and industrial processes for producing the same. Furthermore, it discloses a process for producing Form I of Dolasetron base.

### BACKGROUND AND PRIOR ART

Dolasetron is an antinauseant and antiemetic agent. It is a selective serotonin 5-HT₃ receptor antagonist and is indicated for the prevention of nausea and vomiting associated with emetogenic cancer chemotherapy. Dolasetron is a well-tolerated drug with few side effects.

Synthesis of Dolasetron base is not very widely reported in literature. However, EP0266730/U.S. Pat. No. 4,906,755 describes process for the preparation endo-hexahydro-8-(3-indolylcarbonyloxy)-2,6-methano-2H-quinolizin-3(4H)-one methanesulfonate or Dolasetron mesylate, by the condensation of diethyl malonate with cis-1,4-dichloro-2-butene (2) in presence of lithium hydride in dimethylformamide to give diethyl-3-cyclopentene-1,1-dicarboxylate (3), which on hydrolysis and decarboxylation gave 3-cyclopentene-1-carboxylic acid (4). The compound (4) was further treated with thionyl chloride and pyridine in ethanol to obtain ethyl 3-cyclopentene-1-carboxylate (5). Compound (5) was oxidized to 4-ethoxycarbonyl-1,2-cyclopentanediol (6) by using N-methylmorpholine N-oxide in the presence of osmium tetroxide catalyst. The diol (6) was cleaved to the β-ethoxycarbonylglutaraldehyde (7) using sodium periodate and used directly in the next reaction. Robinson-Schopf cyclisation of the compound (7) with potassium hydrogen phthalate, acetonedicarboxylic acid and glycine ethyl ester hydrochloride resulted in the pseudopelletierine derivative i.e. 7-ethoxycarbonyl-9-(ethoxycarbonylmethyl)-9-azabicyclo-[3.3.1]nonan-3-one (8). The ketone group of compound (8) was reduced with sodiumborohydride in ethanol to give 7-ethoxycarbonyl-9-(ethoxycarbonylmethyl)-9-azabicyclo-[3.3.1]nonan-3-ol (9). The reduced alcohol (9) was treated with dihydropyran to protect the hydroxyl group as a tetrahydropyranyl ether (10). Dieckmann cyclisation of the compound (10) using strong base (potassium t-butoxide) followed by aqueous acid hydrolysis and decarboxylation gave the desired alcohol. The resulting alcohols can exist in two conformations—axial and equatorial. The main product obtained by above procedure was the axial alcohol or endo-hexahydro-8-hydroxy-2,6-methano-2H-quinolizin-3-(4H)-one (11) and it can be separated from the equatorial isomer by crystallization of the camphorsulfonate or tetrafluoroborate salt. The tetrafluoroborate salt of endo-hexahydro-8-hydroxy-2,6-methano-2H-quinolizin-3-(4H)-one (11) was further reacted with 3-indolecarboxylic acid chloride in presence of silver tetrafluoroborate in anhydrous nitroethane at −78° C. to endo-hexahydro-8-(3-indolylcarbonyloxy)-2,6-methano-2H-quinolizin-3(4H)-one or Dolasetron base, which was further converted into Dolasetron mesylate monohydrate (Scheme I) with a yield of 66%. No further purification is described.

The above process uses column chromatography for purification of compounds (9) and (10), which is expensive, time consuming and impractical on an industrial scale. The above patent does not disclose the yield and purity of Dolasetron mesylate obtained and so also for the intermediates. In addition, Osmium tetroxide used for preparation of compound (6) is toxic, has a corrosive action on eyes and hence difficult to use at industrial scale. Also this process uses high volume of water during preparation of the compound (8); preparation of compound (II) from compound (10) is tedious, because the workup involves several extractions with ethyl acetate and preparation of compound (I) in presence of silver tetrafluoroborate involves the use of expensive silver compound.

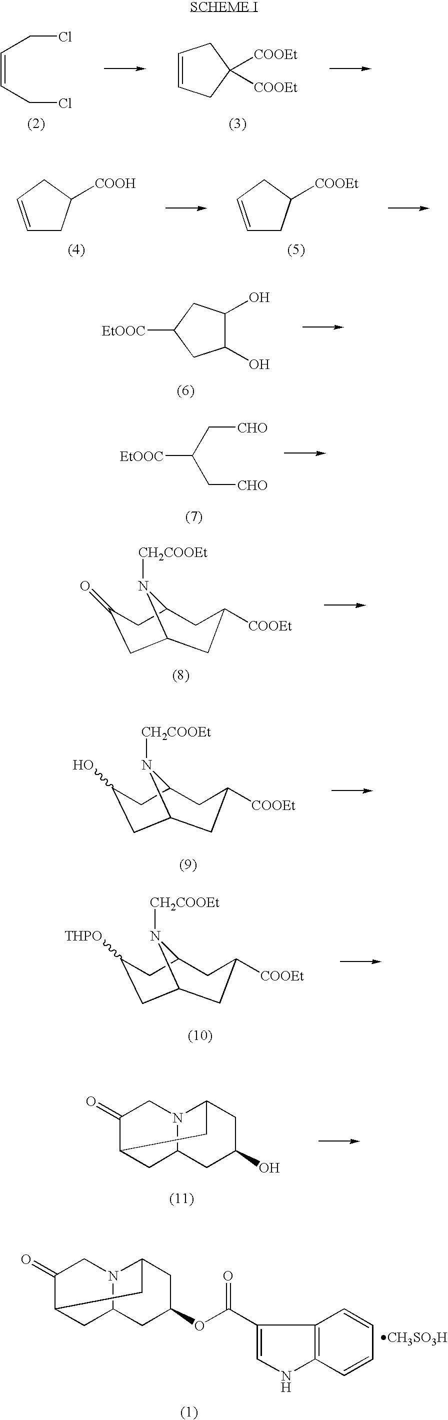

Another method described in EP0339669 provides a process for the preparation of endo-hexahydro-8-(3-indolylcarbonyloxy)-2,6-methano-2H-quinolizin-3(4H)-one methanesulfonate or Dolasetron mesylate (1) by the condensation of dimethyl malonate with cis-1,4-dichloro-2-butene (2) in presence of lithium hydride in dimethyl formamide to give dimethyl-3-cyclopentene-1,1-dicarboxylate (12), which was decarbomethylated to obtain methyl-3-cyclopentene-1-carboxylate (13). This compound (13) was treated with m-chloroperbenzoic acid in dichloromethane to obtain 1-methoxycarbonyl-3-cyclopenteneoxide (14). The compound (13) on ozonolysis gave β-methoxycarbonylglutaraldehyde (15) or the epoxide (14) was reacted with periodic acid to obtain the β-methoxycarbonylglutaraldehyde (15), which was used directly in the next reaction. Robinson-Schopf cyclisation of the compound (15) with potassium hydrogen phthalate, acetonedicarboxylic acid and glycine ethyl ester hydrochloride gave the pseudopelletierine derivative i.e. 7-methoxycarbonyl-9-(methoxycarbonylethyl)-9-azabicyclo[3.3.1]nonan-3-one (16). The ketone group of compound (16) was reduced with sodiumborohydride in methanol to give 7-methoxycarbonyl-9-(methoxycarbonylmethyl)-9-azabicyclo-[3.3.1]nonan-3-ol (17). The reduced alcohol (17) was treated with dihydropyran to protect the hydroxyl group as a tetrahydropyranyl ether (18a) or treated with methylal to protect the hydroxyl group to obtain 3-methoxymethoxy-7-methoxycarbonyl-9-(methoxycarbonylmethyl)-9-azabicyclo[3.3.1]nonan-3-ol (18b).

Dieckmann cyclisation of the compound (18) using strong base (potassium t-butoxide) followed by aqueous acid hydrolysis and decarboxylation gave the endo-hexahydro-8-hydroxy-2,6-methano-2H-quinolizin-3-(4H)-one (11). The alcohol (11) was further reacted with 3-indolecarboxylic acid in presence of trifluoroacetic anhydride in dichloromethane to endo-hexahydro-8-(3-indolylcarbonyloxy)-2,6-methano-2H-quinolizin-3(4H)-one or Dolasetron base (A), which was then converted into Dolasetron mesylate (1) (not shown in Scheme II) by treating with methanesulphonicacid in acetone (Scheme II).

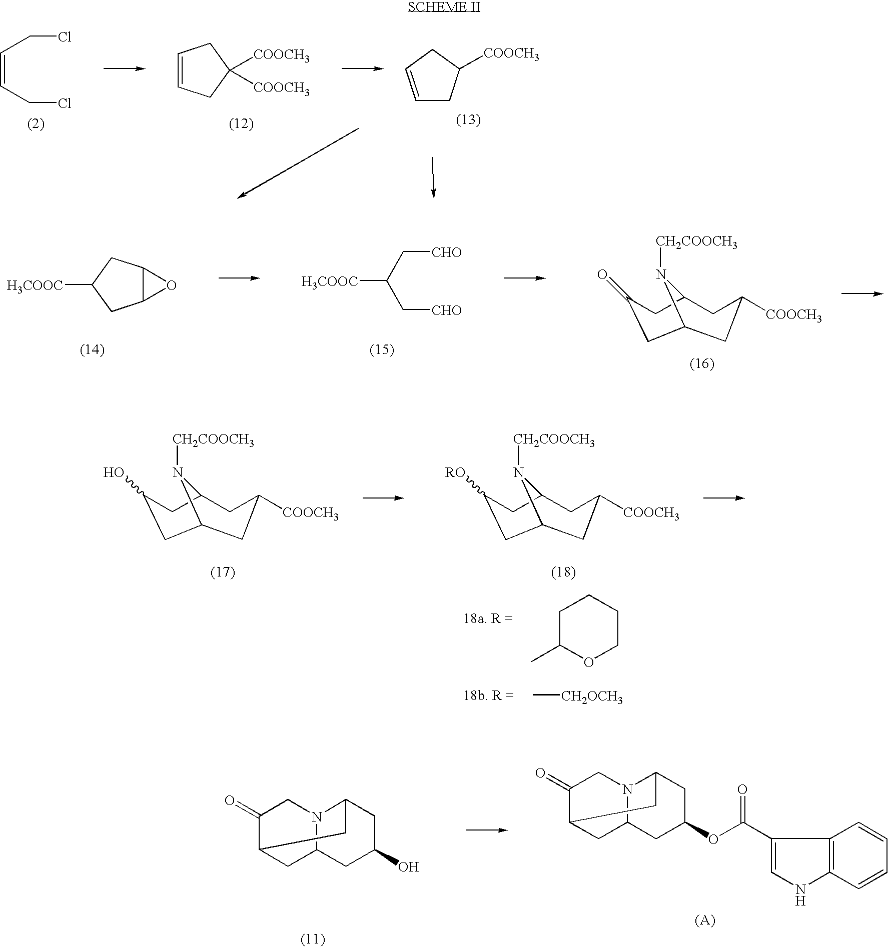

Disadvantages of this process are:
(i) use of high volume of water for preparation of compound (16) and
(ii) preparation of compound (11) from compound (18) which is tedious because at the time of workup, ethyl acetate extractions take up longer period (20 h).

The process is not only time consuming but also expensive on an industrial scale. The patent does not disclose purity of Dolasetron base obtained nor for any of the intermediates.

The process as described in EP 0266730 involves treatment of endo-hexahydro-8-(3-indolylcarbonyloxy)-2,6-methano-2H-quinolizin-3(4H)-one (Dolasetron base) with a solution of methane sulfonic acid in ethanol to provide Dolasetron mesylate monohydrate. EP 0339669 describes crystallization of crude Dolasetron mesylate by dissolution in aqueous isopropanol and regeneration by adding ether. The polymorphic form obtained by the processes described in U.S. Pat. No. 4,906,755/EP 0266730 and EP 0339669 is designated herein as Dolasetron mesylate Form I.

The ability of the compound to exhibit more than one orientation or conformation of molecule within the crystal lattice is called polymorphism. Many organic compounds including active pharmaceutical ingredients (API's) exhibit polymorphism.

Drug substance existing in various polymorphic forms differs from each other in terms of stability, solubility, compressibility, flowability and spectroscopic properties, thus affecting dissolution, bioavailability and handling characteristics of the substance.

Rate of dissolution of an API's in patient's stomach fluid can have therapeutic consequences since it imposes an upper limit on the rate at which an orally administrated API can reach the patient bloodstream. Flowability affects the ease with which the material is handled while processing a pharmaceutical product.

Investigation of crystal polymorphism is an essential step in pharmaceutical research due to the influence of solid-state properties on dosage form.

As the polymorphs are known to possess different spectroscopic properties, technique such as X-Ray powder diffraction (XRPD), Fourier transformer Infrared (FT-IR) spectroscopy, Solid State ¹³C-NMR, and thermal method of analysis are keys to identify and characterize the new polymorphs or existing polymorphs.

The discovery of new polymorphs with same or better pharmaceutical equivalence and bioequivalence as that of the known polymorphs provides an opportunity to improve the performance characteristic of the pharmaceutical product.

The prior art describes isolation of endo-hexahydro-8-(3-indolylcarbonyloxy)-2,6-methano-2H-quinolizin-3(4H)-one or Dolasetron base as an oil. It is desirable to have the product in the solid form than oil, as solid is easy to handle and easy to purify.

Dolasetron base is isolated as a solid in EP 0339669. However, there is no evidence of polymorphism.

WO2006056081 discloses purification of Dolasetron base using strong acid especially methanesulphonic acid in presence of acid halide.

In our endeavour to develop a process for the preparation of Dolasetron base, we have surprisingly discovered novel polymorphic forms of Dolasetron base.

### OBJECTS OF THE INVENTION

An object of the disclosure is to provide a simple, economical and industrial process for the preparation of endo-hexahydro-8-(3-indolylcarbonyloxy)-2,6-methano-2H-quinolizin-3(4H)-one or Dolasetron base.

One more object of the disclosure is to provide a simple, economical and industrial process for the preparation of substantially pure endo-hexahydro-8-(3-indolylcarbonyloxy)-2,6-methano-2H-quinolizin-3(4H)-one methanesulfonate or Dolasetron mesylate.

Another object of the disclosure is to provide a process to prepare Form I of Dolasetron base.

Yet another object is to provide novel polymorphic forms of Dolasetron base having improved stability, compressibility and bioavailability and industrial processes for producing them.

### SUMMARY OF THE INVENTION

Accordingly, the present disclosure provides a process for producing endo-hexahydro-8-(3-indolylcarbonyloxy)-2,6-methano-2H-quinolizin-3(4H)-one (Dolasetron base) having structural formula (A).

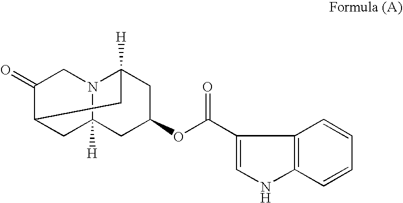

In one aspect, the present invention provides a process for producing endo-hexahydro-8-(3-indolylcarbonyloxy)-2,6-methano-2H-quinolizin-3(4H)-one mesylate (Dolasetron mesylate) having structural formula (I).

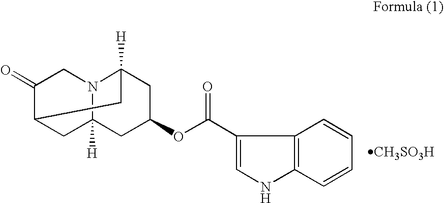

In another aspect, the present disclosure provides a process for producing polymorphic Form I of Dolasetron base.

In one aspect, the present invention provides a crystalline polymorphic Form II of endo-hexahydro-8-(3-indolylcarbonyloxy)-2,6-methano-2H-quinolizin-3(4H)-one (Dolasetron base).

In another aspect, the present invention relates to a process for producing the polymorphic Form II of Dolasetron base.

In yet another aspect, the present invention provides a crystalline polymorphic Form III of endo-hexahydro-8-(3-indolylcarbonyloxy)-2,6-methano-2H-quinolizin-3(4H)-one (Dolasetron base).

In one aspect, the present invention relates to a process for producing polymorphic Form III of Dolasetron base.

In yet another aspect, the present invention provides a crystalline polymorphic Form IV of endo-hexahydro-8-(3-indolylcarbonyloxy)-2,6-methano-2H-quinolizin-3(4H)-one (Dolasetron base).

In a further aspect, the present invention relates to a process for producing polymorphic Form IV of Dolasetron base.

In one aspect, the present invention provides a crystalline polymorphic Form V of endo-hexahydro-8-(3-indolylcarbonyloxy)-2,6-methano-2H-quinolizin-3(4H)-one (Dolasetron base).

In another aspect, the present invention relates to a process for producing polymorphic Form V of Dolasetron base.

### BRIEF DESCRIPTION OF ACCOMPANYING DRAWINGS

FIG. 1 shows XRPD pattern of Dolasetron base Form I

FIG. 2 shows DSC plot of Dolasetron base Form I

FIG. 3 shows FT-IR spectrum of Dolasetron base Form I

FIG. 4 shows XRPD pattern of Dolasetron base Form II

FIG. 5 shows DSC plot of Dolasetron base Form II

FIG. 6 shows FT-IR spectrum of Dolasetron base Form II

FIG. 7 shows XRPD pattern of Dolasetron base Form III

FIG. 8 shows DSC plot of Dolasetron base Form III

FIG. 9 shows FT-IR spectrum of Dolasetron base Form III

FIG. 10 shows XRPD pattern of Dolasetron base Form IV

FIG. 11 shows DSC plot of Dolasetron base Form IV

FIG. 12 shows FT-IR spectrum of Dolasetron base Form IV

FIG. 13 shows XRPD pattern of Dolasetron base Form V

FIG. 14 shows DSC plot of Dolasetron base Form V

FIG. 15 shows FT-IR spectrum of Dolasetron base Form V

### DETAILED DESCRIPTION OF THE INVENTION

The present disclosure provides a process for the preparation of endo-hexahydro-8-(3-indolylcarbonyloxy)-2,6-methano-2H-quinolizin-3(4H)-one having structural formula (A) (Dolasetron base) comprising:

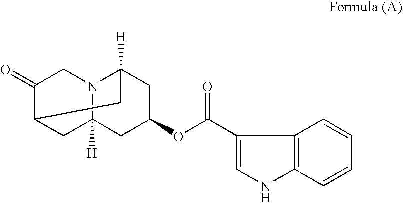

a) reacting a compound having structural formula (4)

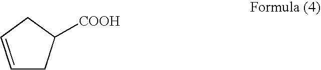

with thionyl chloride or hydrochloric acid in an alcohol to obtain a compound having structural formula (V);

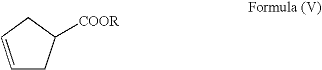

b) treating the compound (V) with m-chloroperbenzoic acid in a solvent to form an epoxide compound having structural formula (XIX);

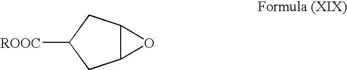

c) treating the compound (XIX) with periodic acid to obtain a compound having structural formula (VII);

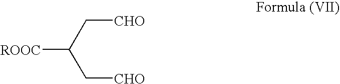

d) cyclising the compound (VII) using potassium hydrogen phthalate, acetonedicarboxylic acid and glycine ester hydrochloride in water to obtain a pseudopelletierine derivative having formula (VIII);

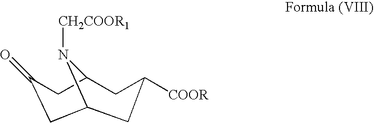

e) reducing the compound (VIII) with sodiumborohydride in an alcohol and treating it with an organic acid to obtain a compound having structural formula (IX);

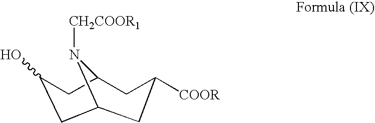

f) treating the compound (IX) with a silyl reagent, in an organic solvent to form silyl derivative having structural formula (XX), wherein Z is a silyl group;

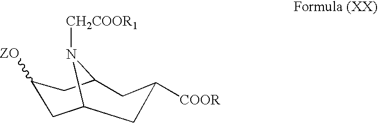

g) treating the compound (XX) with a strong base in toluene and further treating it with a mixture of organic acid and organic solvent to form a compound having structural formula (XXI);

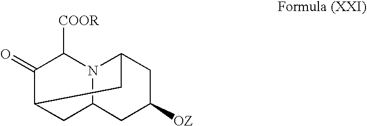

h) treating the compound (XXI) with an inorganic acid in water and treating it with an organic solvent to give a compound having structural formula (II); and

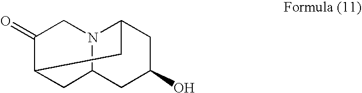

i) reacting the compound (II) with indole-3-carboxylic acid in presence of trifluoroacetic acid anhydride to obtain Dolasetron base of structural formula (A);
   wherein, R is Et, Me, or OCH₂Ph; R₁ is Et, Me, or OCH₂Ph; and Z is selected from trimethyl silyl, isopropyl dimethyl silyl, t-butyldimethyl silyl, t-butyldiphenyl silyl, tribenzyl silyl, and triisopropyl silyl.

The present disclosure also provides a process for preparation of Dolasetron mesylate or endo-hexahydro-8-(3-indolylcarbonyloxy)-2,6-methano-2H-quinolizin-3(4H)-one methanesulfonate of structural formula (1) in high yield and substantial purity comprising:

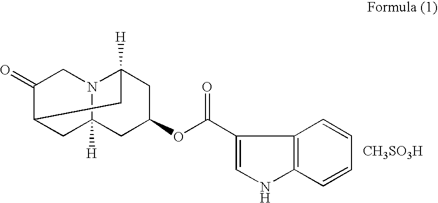

a) converting the Dolasetron base of structural formula (A) into its mesylate salt by treating with methane sulphonic acid in a suitable organic solvent; and
b) purifying the Dolasetron mesylate by treating with a base and further adding methanesulphonic acid to obtain highly pure compound of structural formula (I).

The organic solvent is selected from alcohols such as methanol, ethanol, and isopropanol, halogenated solvents such as dichloromethane and chloroform, ketones such as acetone and methyl ethyl ketone or mixture thereof, preferably acetone.

The base is selected from sodium carbonate, sodium hydroxide, potassium hydroxide, and potassium carbonate, preferably sodium carbonate.

The Dolasetron base used for preparing its acid addition salt is selected from Dolasetron base Form I, Dolasetron base Form II, Dolasetron base Form III, Dolasetron base Form IV and Dolasetron base Form V.

In accordance to Scheme III, a process for the preparation of 3-cyclopentene-1-carboxylic acid ester (5) is disclosed, said process comprising: reacting 3-cyclopentene-1-carboxylic acid (4) with anhydrous HCl gas or concentrated hydrochloric acid or thionyl chloride in an alcohol, wherein the alcohol is either methanol or ethanol; treating the compound (5) with m-chloroperbenzoic acid in a solvent selected from dichloromethane, toluene and ethyl acetate to obtain the corresponding epoxide (19); reacting the compound (19) with periodic acid under nitrogen atmosphere to obtain compound (7); treating the compound (7) with potassium hydrogen phthalate, acetonedicarboxylic acid and glycine ester hydrochloride in water to obtain pseudopelletierine derivative (8); reducing the compound (8) with sodiumborohydride in an alcohol and further treating with an organic acid to obtain compound (9), wherein the organic acid is selected from formic acid, methane sulphonic acid and acetic acid; treating the compound (9) with silyl halide in presence of imidazole in an organic solvent to obtain compound (20), wherein the organic solvent is selected from ketones, esters and ethers, preferably from acetone, tetrahydrofuran, 1,4-dioxane, dichloromethane, chloroform, N,N-dimethyl formamide, ethyl acetate and acetonitrile.

Scheme III given below shows the complete process as a flow chart:

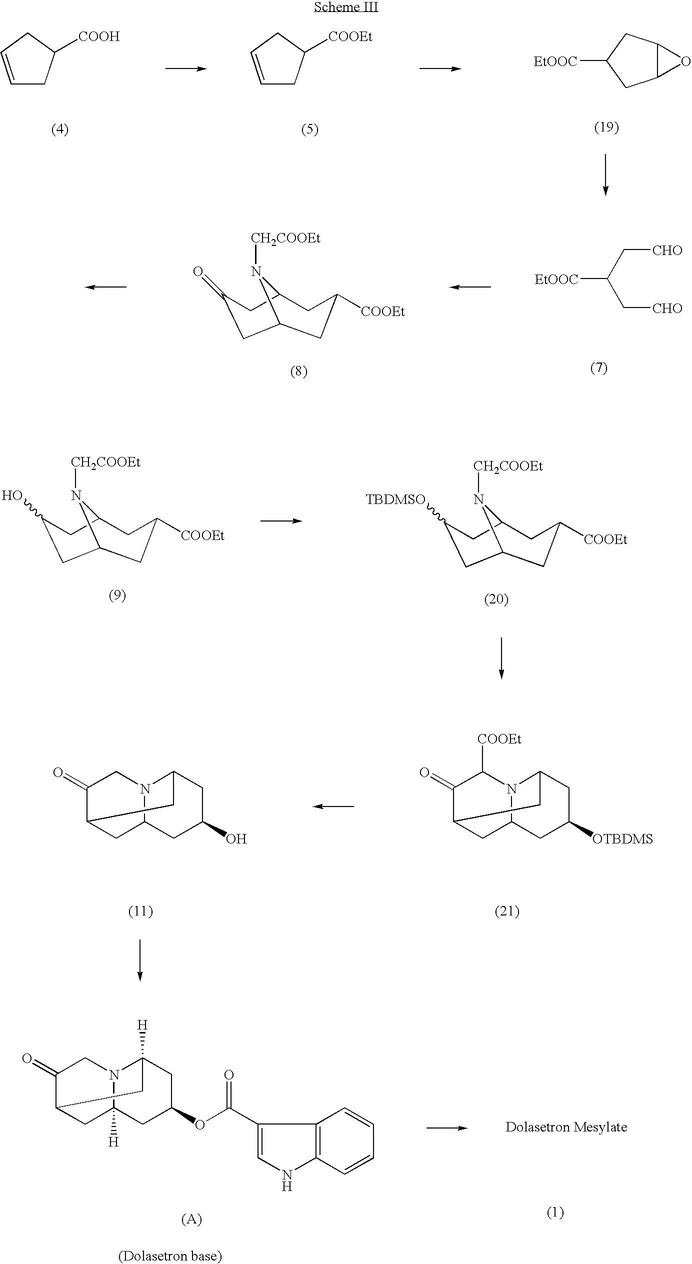

A major advantage of the use of silyl protecting group is that it yields greater than 95% of compound (20) as compared to, use of other protecting groups such as dihydropyran (75%) or methylal (84%).

The compound (20) is treated with a strong base in toluene and further treated with an organic acid in an organic solvent to form compound (21). The organic solvent is selected from halogenated solvents, ethers and esters. The organic solvent is preferably selected from methylene chloride, chloroform, ethyl acetate, isopropyl acetate, diethyl ether and diisopropyl ether, or mixture thereof. The organic acid is selected from formic acid and acetic acid.

The compound (21) is heated with hydrochloric acid in water to give compound (II). Hydrochloric acid and water are used in the ratio of 1:2 volumes. The ratio of compound (21) to water in the reaction is about 1:8 to 1:10. The reaction mixture is concentrated and the residue obtained is treated with an organic solvent and filtered. The filtrate is concentrated to obtain compound (11). The organic solvent is selected from alcohols and halogenated solvent preferably methanol, ethanol, isopropanol, n-butanol, dichloromethane, chloroform or mixture thereof. The reaction mixture is extracted with an organic solvent selected from ethylacetate, isopropanol or n-butanol. Alternately the reaction mixture is saturated with an inorganic salt and extracted with an organic solvent selected from ethylacetate or n-butanol or isopropanol.

The compound (11) is reacted with indole-3-carboxylic acid in presence of trifluoroacetic acid anhydride in dichloromethane to give Dolasetron base. The ratio of indole-3-carboxylic acid and trifluoro acetic anhydride used is in the range of 1:1.1 to 1:2. Dolasetron base thus obtained is isolated by conventional method. Dolasetron base is solubilized in acetone and converted into its mesylate salt using methane sulphonic acid. The resultant mesylate salt is dissolved in water and extracted with a halogenated solvent or ester to remove traces of impurity. The halogenated solvent is selected from dichloromethane and chloroform, and the ester is selected from methyl acetate, ethyl acetate and isopropyl acetate. The aqueous layer is basified with a base to obtain Dolasetron base. The base is selected from sodium carbonate, potassium carbonate, sodium bicarbonate, potassium bicarbonate, sodium hydroxide and potassium hydroxide or mixture thereof. Dolasetron base thus obtained is treated with methane sulphonic acid in a mixture of acetone and water to provide Dolasetron mesylate.

### Process for Preparing Dolasetron Base Form I

The present disclosure teaches a process for preparation of Form 1 of Dolasetron base or endo-hexahydro-8-(3-indolylcarbonyloxy)-2,6-methano-2H-quinolizin-3(4H)-one.

The process for producing polymorphic Form I of endo-hexahydro-8-(3-indolylcarbonyloxy)-2,6-methano-2H-quinolizin-3(4H)-one (Dolasetron base), comprises dissolving Dolasetron base in a solubilizing solvent at a temperature in the range of about 25° C. to 90° C. and optionally adding anti-solvent to precipitate into a solid.

Dolasetron base is dissolved in a solubilizing solvent selected from aliphatic ketones, aliphatic nitriles, aliphatic esters and cyclic ethers, or mixture thereof at a temperature in the range of about 25° C. to 90° C. to get a clear solution. The aliphatic ketone is acetone, aliphatic nitrile is acetonitrile, aliphatic ester is ethyl acetate, and the cyclic ether is tetrahydrofuran (THF) or 1,4-dioxane. The dissolved solid is obtained by cooling the solution at a temperature range of about 0° C. to 20° C. or alternately by addition of miscible anti-solvent. The anti-solvent is selected from a group consisting of aliphatic hydrocarbons and aliphatic ethers, or mixture thereof. The anti-solvent is selected from diethyl ether (DEE), diisopropyl ether (DIPE), n-hexane and n-heptane, or mixture thereof. When the ketone is used as a solvent, the antisolvent used is aliphatic hydrocarbon.

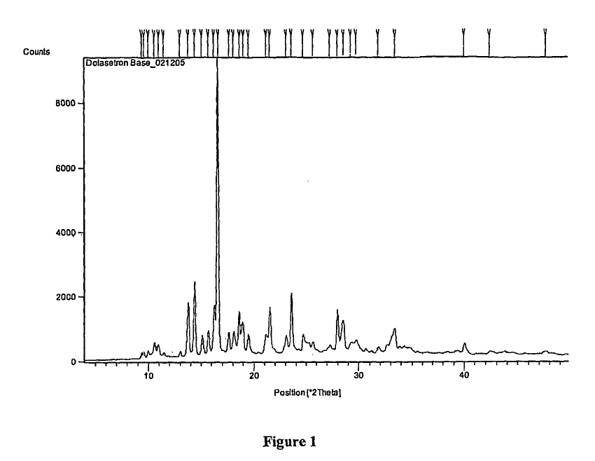

The XRPD of Dolasetron base Form I exhibit following peaks. (FIG. 1):

| Form I | |
|---|---|
| Position [°2θ] | Rel. Int. [%] |
| 9.3724 | 2.42 |
| 9.5704 | 2.56 |
| 9.9972 | 2.88 |
| 10.5758 | 5.98 |
| 11.0164 | 4.05 |
| 11.4712 | 2.01 |
| 13.0348 | 2.26 |
| 13.7964 | 19.93 |
| 14.4223 | 25.94 |
| 15.1141 | 7.78 |
| 15.6909 | 8.99 |
| 16.2557 | 18.51 |
| 16.6375 | 100.00 |
| 17.6823 | 7.85 |
| 18.1145 | 9.06 |
| 18.6538 | 15.79 |
| 19.0321 | 10.48 |
| 19.4831 | 7.46 |
| 21.1547 | 7.35 |
| 21.5435 | 16.27 |
| 23.0633 | 6.83 |
| 23.5910 | 22.17 |
| 24.7576 | 7.08 |
| 25.6954 | 4.24 |
| 27.3049 | 2.78 |
| 28.0013 | 15.62 |
| 28.6021 | 10.78 |
| 29.2609 | 3.55 |
| 29.7655 | 4.45 |
| 31.8342 | 1.41 |
| 33.4519 | 6.94 |
| 40.0693 | 3.85 |
| 42.3787 | 0.84 |
| 47.6761 | 1.17 |

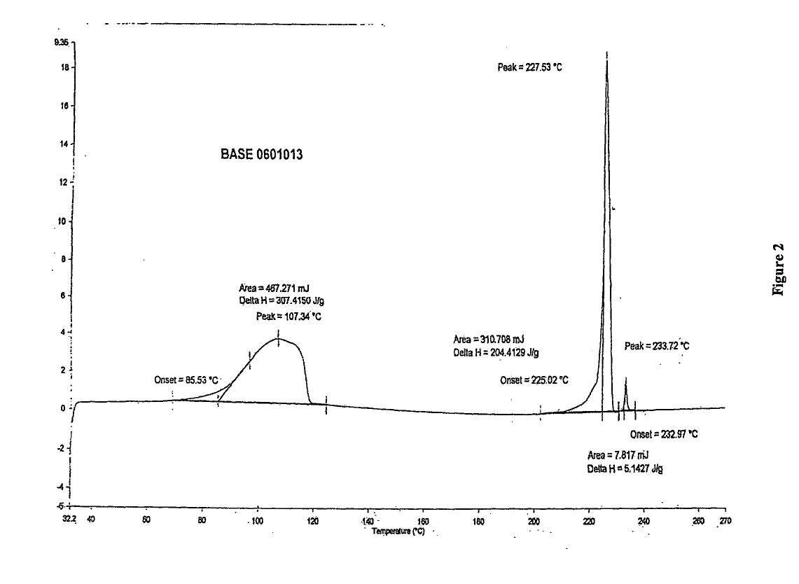

DSC of Dolasetron base Form I show two characteristic exotherms at 97° C. and 228° C. (FIG. 2).

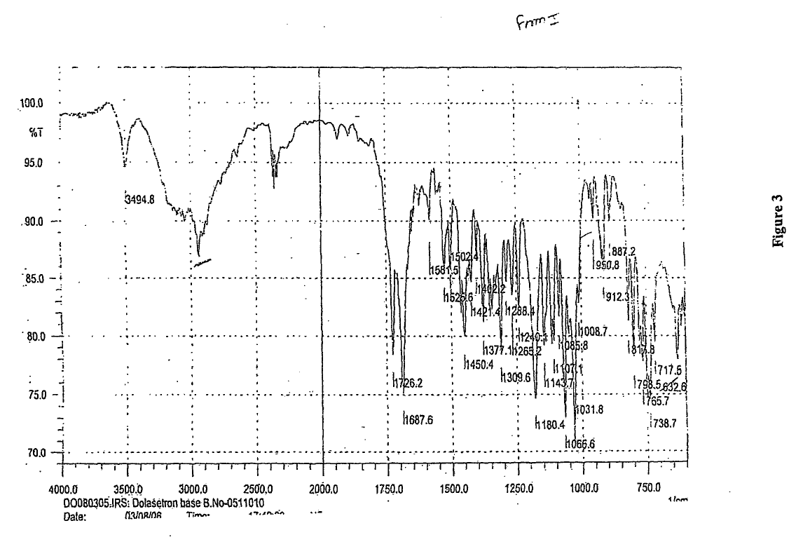

FT-IR spectrum of Dolasetron base Form I shows characteristic absorption at 3494, 1726, 1687, 1581, 1525, 1450, 1309, 1288, 1265, 1180, 1107, 1066, 1031, 950, 798, 765, 738, 717 cm⁻¹ (FIG. 3).

Further, the present invention provides novel polymorphic forms of Dolasetron base viz. Form II, Form III, Form IV and Form V and processes for producing the same.

### Dolasetron Base Form II

Dolasetron base is dissolved in aliphatic ethers at a temperature between about 60° C. to 80° C., preferably about 70° C. to 80° C. The clear solution is cooled to a temperature between about −5° C. to 20° C., preferably about 2° C. to 7° C. The suspension was stirred at the same temperature for 2 hr. The separated solid is isolated by filtration and dried at about 50° C.-70° C. to obtain the crystalline product.

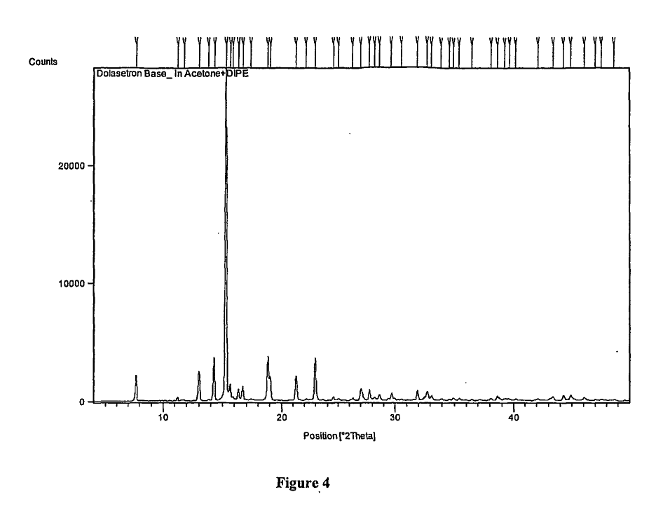

Another process for producing the polymorphic Form II of Dolasetron base, comprises: dissolving Dolasetron base in acetone at a temperature ranging between about 20° C. and 40° C. and adding diisopropyl ether to obtain the crystalline product. The XRPD of Dolasetron base Form II exhibits following peaks (FIG. 4):

| Form II | |
|---|---|
| Position [°2θ] | Rel. Int. [%] |
| 7.6951 | 7.32 |
| 11.2107 | 1.03 |
| 11.7873 | 0.31 |
| 13.0175 | 8.91 |
| 13.7774 | 0.34 |
| 14.2745 | 12.85 |
| 15.2884 | 100.00 |
| 15.6226 | 4.99 |
| 16.2987 | 3.17 |
| 16.6824 | 4.09 |
| 17.3849 | 0.49 |
| 18.8272 | 12.90 |
| 21.2776 | 7.55 |
| 22.1677 | 0.59 |
| 22.9362 | 12.67 |
| 24.5293 | 1.26 |
| 24.9950 | 0.68 |
| 26.2557 | 0.85 |
| 26.9843 | 3.63 |
| 27.7523 | 3.06 |
| 28.1915 | 1.15 |
| 28.6294 | 1.89 |
| 29.6937 | 2.38 |
| 31.9082 | 3.19 |
| 32.7395 | 2.81 |
| 33.1051 | 1.56 |
| 33.9005 | 0.61 |
| 34.5350 | 0.46 |
| 34.8785 | 0.81 |
| 35.3815 | 0.71 |
| 36.4598 | 0.50 |
| 38.0611 | 0.70 |
| 38.5883 | 1.24 |
| 39.2167 | 0.71 |
| 39.6245 | 0.61 |
| 40.1352 | 0.56 |
| 42.0607 | 0.54 |
| 43.3383 | 1.32 |
| 44.2455 | 1.59 |
| 44.8736 | 1.74 |
| 46.0532 | 1.10 |
| 47.0090 | 0.43 |
| 47.5326 | 0.46 |
| 48.6384 | 0.37 |

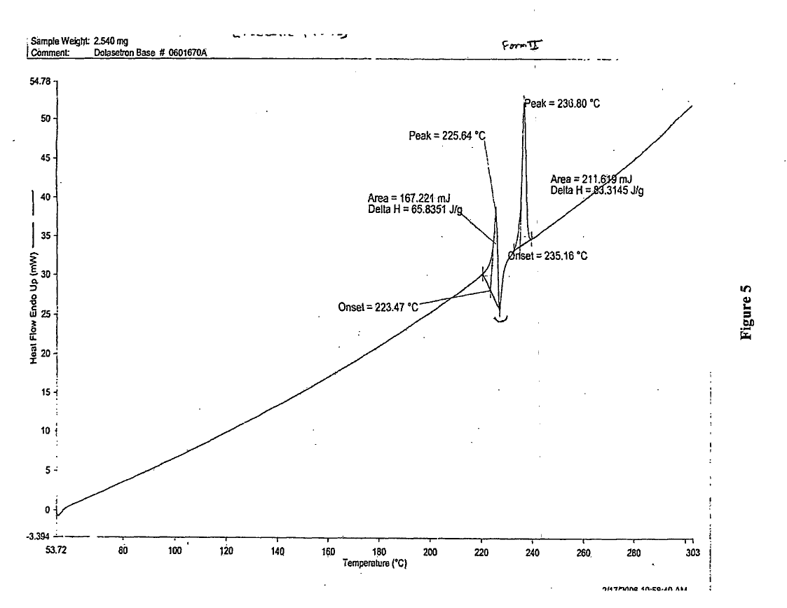

DSC of Dolasetron base Form II shows two endotherms, one at 225° C. followed by a second at 236° C. (FIG. 5).

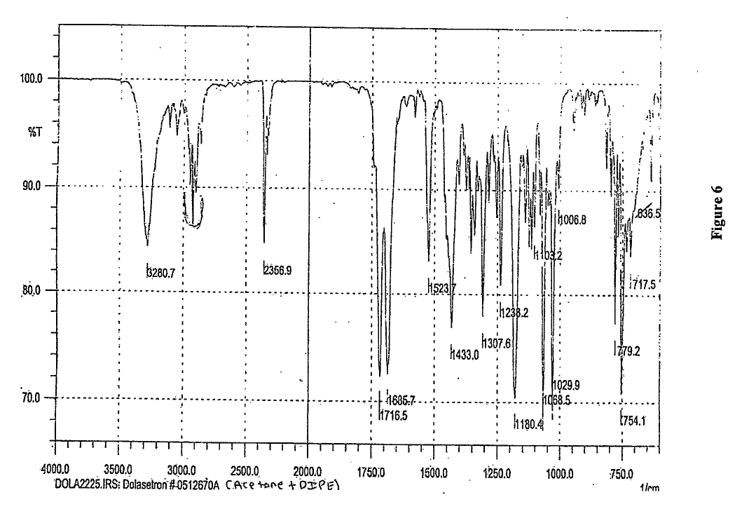

FT-IR spectrum of Dolasetron base Form II shows characteristic absorption at 3280, 1716, 1685, 1523, 1433, 1307, 1238, 1180, 1068, 1029, 79, 754, 717 cm⁻¹ (FIG. 6):

### Dolasetron Base Form III

Dolasetron base is dissolved in a solubilizing solvent like in aliphatic alcohols or in polar aprotic solvent selected from N,N-dimethyl formamide (DMF), dimethyl sulfoxide (DMSO), N,N-dimethyl acetamide (DMA), or cyclic ethers selected from tetrahydrofuran (THF) and 1,4-dioxane or mixture thereof at a temperature range of about 25° C. to 30° C. The clear solution is cooled and the solid is obtained by adding water as an anti-solvent. The separated solid is then isolated by filtration and dried at about 50° C. to 70° C. to obtain crystalline Dolasetron base Form III.

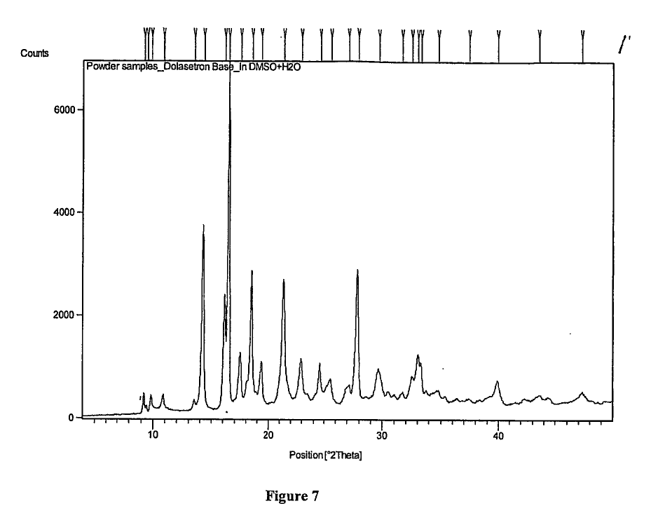

In another process, Dolasetron base is dissolved in a solubilizing solvent like in cyclic ethers selected from tetrahydrofuran (THF) and 1,4-dioxane, in aliphatic esters selected from ethyl acetate, in aliphatic alcohols selected from n-propanol and isopropanol, or mixture thereof at a temperature in the range of about 70° C. to 110° C. The clear solution is cooled and the separated solid is then isolated by filtration and dried at about 50° C. to 70° C. to obtain crystalline Dolasetron base Form III. The XRPD of Dolasetron base Form III exhibits following peaks (FIG. 7):

| Form III | |
|---|---|
| Position [°2θ] | Rel. Int. [%] |
| 9.2394 | 6.23 |
| 9.4668 | 2.55 |
| 9.8411 | 5.69 |
| 10.8592 | 5.90 |
| 13.4933 | 3.83 |
| 14.2878 | 53.97 |
| 16.1274 | 35.49 |
| 16.5113 | 100.00 |
| 17.5210 | 17.30 |
| 18.4973 | 42.30 |
| 19.3556 | 15.04 |
| 21.3917 | 38.94 |
| 22.9458 | 15.00 |
| 24.6119 | 13.93 |
| 25.5599 | 9.05 |
| 27.1031 | 7.03 |
| 27.9029 | 36.54 |
| 29.6730 | 11.88 |
| 31.6704 | 4.45 |
| 32.4906 | 9.14 |
| 33.0179 | 16.04 |
| 33.3242 | 12.53 |
| 34.7579 | 4.73 |
| 37.3862 | 2.06 |
| 39.9142 | 7.12 |
| 43.5379 | 2.22 |
| 47.2834 | 2.91 |

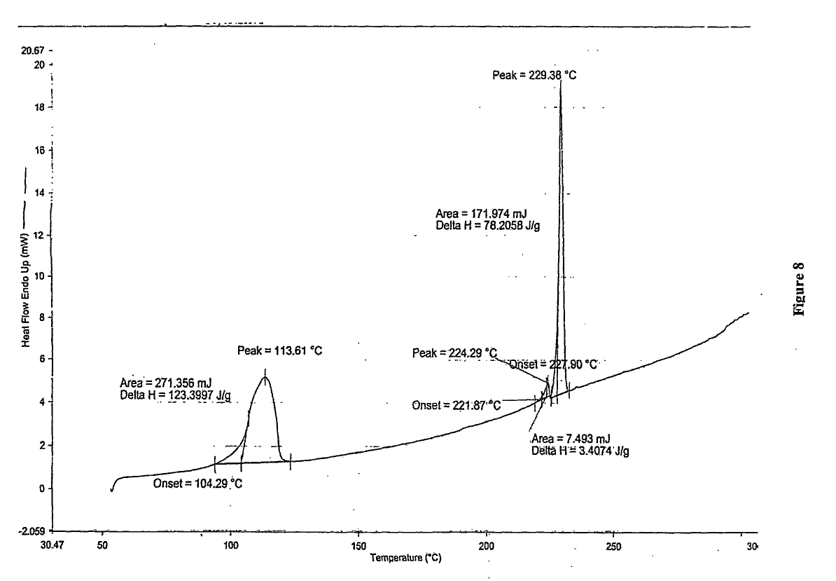

DSC of Dolasetron base Form III shows a first endotherm at 113° C. and the second endotherm at 229° C. (FIG. 8).

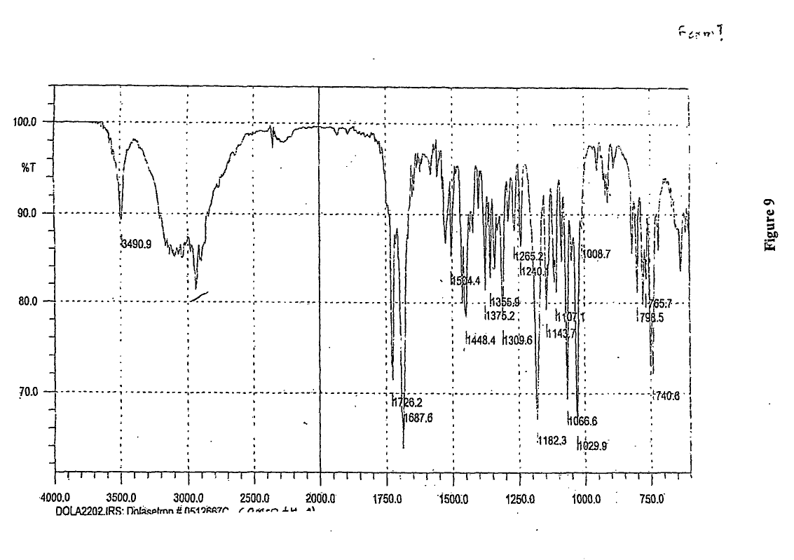

FT-IR spectrum of Dolasetron base Form III shows characteristic absorption at 3490, 1726, 1687, 1504, 1448, 1375, 1309, 1182, 1143, 1066, 1029, 798, 765, 740 cm⁻¹ (FIG. 9).

### Dolasetron Base Form IV

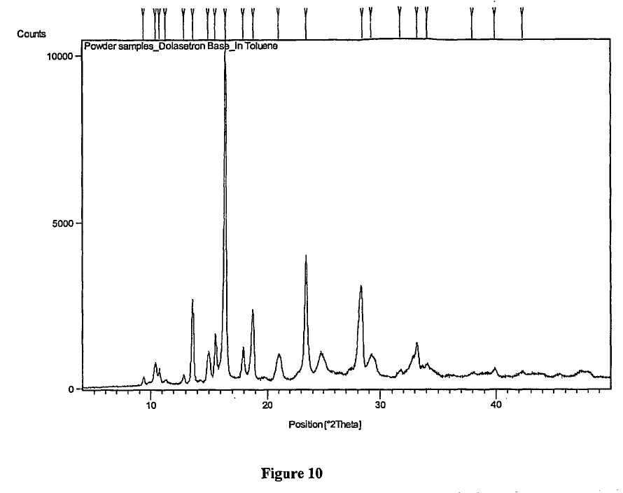

Dolasetron base is dissolved in aromatic hydrocarbons, lower aliphatic alcohols, chlorinated hydrocarbon or mixture thereof at a temperature in the range of about 40° C. to 110° C. The clear solution is allowed to cool and the separated solid is isolated by filtration and dried at about 50° C. to 70° C. to obtain crystalline Dolasetron base Form IV. The aromatic hydrocarbon is toluene; lower aliphatic alcohol is selected from methanol or ethanol; and chlorinated hydrocarbon is chloroform or methylene dichloride (MDC). The XRPD of Dolasetron base Form IV exhibits following peaks (FIG. 10):

| Form IV | |
|---|---|
| Position [°2θ] | Rel. Int. [%] |
| 9.3702 | 2.54 |
| 10.4465 | 6.30 |
| 10.7610 | 4.61 |
| 11.3038 | 1.31 |
| 12.8438 | 2.57 |
| 13.6345 | 24.19 |
| 14.9376 | 8.65 |
| 15.5688 | 14.21 |
| 16.4557 | 100.00 |
| 17.9327 | 9.74 |
| 18.8471 | 16.80 |
| 20.9858 | 7.40 |
| 23.4505 | 35.06 |
| 28.4316 | 20.07 |
| 29.1900 | 6.17 |
| 31.7488 | 1.70 |
| 33.2080 | 9.68 |
| 34.0855 | 3.62 |
| 38.0021 | 1.18 |
| 39.9206 | 2.13 |
| 42.2787 | 1.13 |

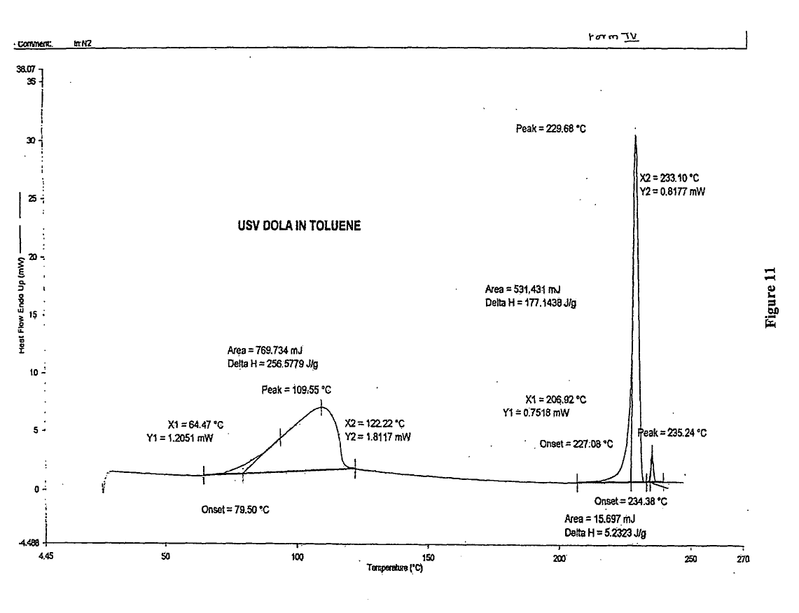

DSC of Dolasetron base Form IV shows a small endotherm at 98° C. followed by two endotherms first at 229.5° C. and second at 235.9° C. (FIG. 11).

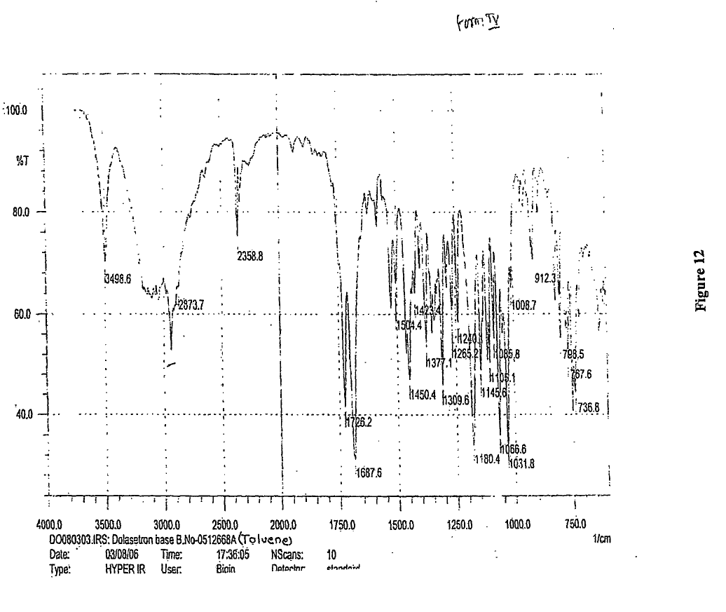

FT-IR spectrum of Dolasetron base Form IV shows characteristic absorption at 3498, 1726, 1687, 1504, 1450, 1377, 1309, 1265, 1240, 1180, 1145, 1105, 1085, 1066, 1031, 912, 798, 767, 736 cm⁻¹ (FIG. 12).

### Dolasetron Base Form V

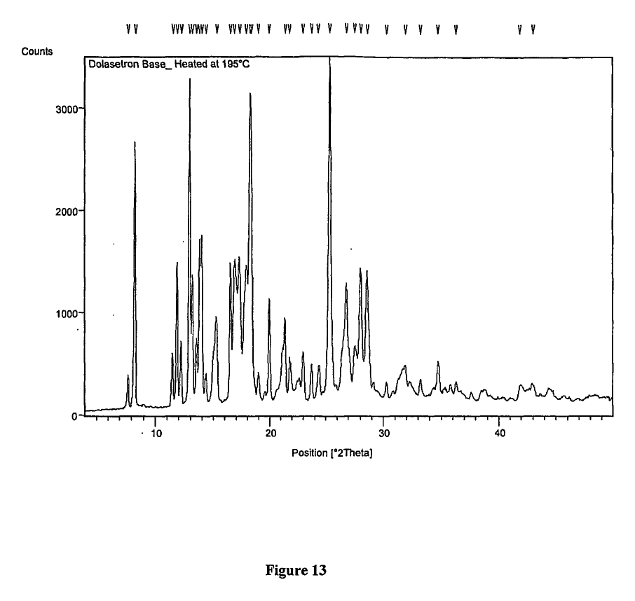

Dolasetron base undergoes solid-state transformation into new crystalline Form V on heating. The heating is generally carried out in the temperature range of about 150° C. to 200° C. The XRPD of Dolasetron base Form V exhibits following peaks (FIG. 13):

| Form V | |
|---|---|
| Position [°2θ] | Rel. Int. [%] |
| 7.7366 | 9.59 |
| 8.3350 | 65.43 |
| 11.4893 | 15.03 |
| 11.8951 | 36.15 |
| 12.2380 | 14.94 |
| 12.9631 | 90.71 |
| 13.1931 | 37.78 |
| 13.5550 | 19.39 |
| 13.8111 | 49.24 |
| 14.0013 | 49.61 |
| 14.3550 | 9.38 |
| 15.3181 | 22.69 |
| 16.5041 | 41.62 |
| 16.8688 | 42.44 |
| 17.3018 | 43.22 |
| 17.9151 | 41.17 |
| 18.2586 | 91.56 |
| 18.3637 | 73.77 |
| 18.9766 | 9.13 |
| 19.9918 | 30.82 |
| 21.4003 | 24.41 |
| 21.8024 | 13.46 |
| 23.0034 | 14.67 |
| 23.7376 | 11.19 |
| 24.3525 | 10.47 |
| 25.3915 | 100.00 |
| 26.8348 | 33.73 |
| 27.5637 | 16.15 |
| 28.0815 | 38.81 |
| 28.6278 | 37.42 |
| 30.2819 | 5.20 |
| 31.8967 | 10.10 |
| 33.2126 | 5.69 |
| 34.7295 | 10.98 |
| 36.2828 | 4.66 |
| 41.8928 | 3.84 |
| 43.0107 | 3.27 |

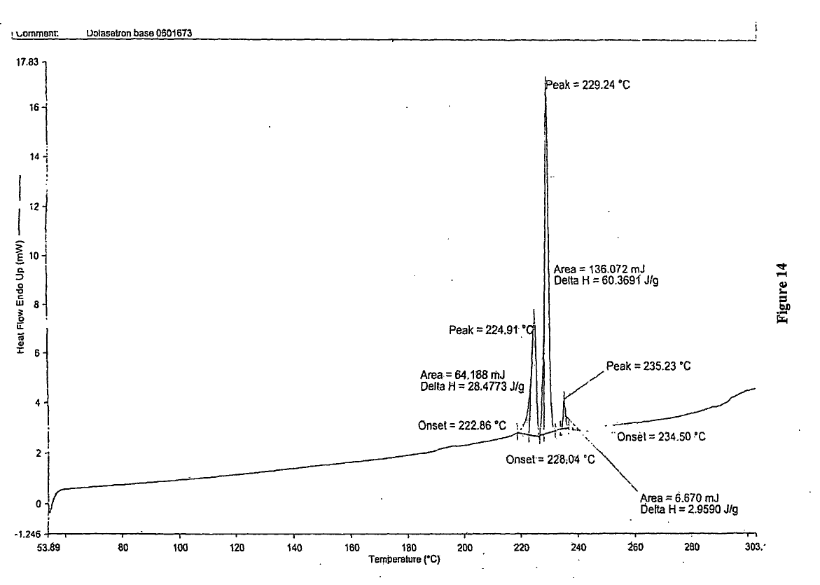

DSC of Dolasetron base Form V shows two endotherms, first at 224° C. followed by the second at 229° C. (FIG. 14).

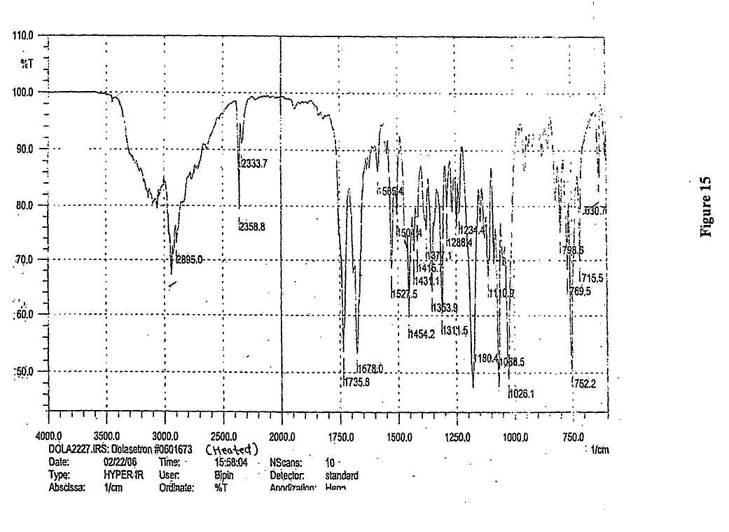

FT-IR spectrum of Dolasetron base Form V shows characteristic absorption at 1735, 1678, 1585, 1527, 1454, 1353, 1311, 1180, 1110, 1068, 1026, 912, 798, 769, 752, 715 cm⁻¹ (FIG. 15).

The crystallization process hitherto described to prepare the novel polymorphs comprises, dissolving Dolasetron base in the selected solvent either with or without heating, preferably with heating at or near boiling point of the solvent. The resultant solution is cooled to about −5° C. to 30° C. for several hours to regenerate the solid. Solid generated either by cooling or by using an anti solvent. The precipitated solids are isolated and dried at about ambient to 70° C. temperature.

The novel polymorphs of Dolasetron base are characterized by X-ray powder diffraction. X-ray powder diffraction pattern has been obtained on Xpert'PRO, Panalytical diffractometer equipped with accelerator detector using Copper Kα (λ=1.5406 Å) radiation with scanning range between 4-50° 2θ at a scanning speed of 2°/min.

The novel polymorphs of Dolasetron base are characterized by Differential Scanning Calorimeter (DSC). The DSC was performed on Perkin Elmer Diamond DSC instrument. Samples of 2 mg to 3 mg weighed in aluminium crucibles with holes were scanned at a heating rate of 5° C. per minute under nitrogen atmosphere at a rate of 35 mL/min from the temperature range 50-250° C.

The novel polymorphs of Dolasetron base are characterized by Fourier-transform infrared (FT-IR) spectroscopy. The FT-IR spectrum was obtained on a FT-IR 8300, Shimadzu instrument, in the range of 4000-400 cm⁻¹ with a resolution of 4 cm⁻¹.

The present invention is described herein below with examples, which are illustrative only and should not be construed to limit the scope of the present invention in any manner.

### EXAMPLES

### Example 1

### Preparation of ethyl-3-cyclopentene-1-carboxylate (5)

A solution of 3-cyclopentene-1-carboxylic acid (500 g, 4.45 mole) in ethanol (500 mL) was stirred at 5-10° C. Then thionyl chloride (257.59 g, 2.16 mole) was added in a drop wise manner for 1 hr. After complete addition was over, the reaction mixture was stirred at room temperature for 30 min. The reaction mixture was poured into the water (1000 mL) and extracted with ethyl acetate (2×250 mL). The ethyl acetate layer was washed with 10% sodium carbonate solution (500 mL), with water (2×500 mL) and concentrated to give ethyl-3-cyclopentene-1-carboxylate (5). Yield: 558 g, 89.42%.

### Example 2

### Preparation of 1-ethoxycarbonyl-3-cyclopenteneoxide (19)

A solution of ethyl-3-cyclopentene-1-carboxylate (5) (1 Kg, 7.13 mole) in dichloromethane (8 L) was stirred at 5-10° C. Then 70% meta-chloroperbenzoic acid (2.4 Kg, 9.73 mole) was added in lots for 1 hr at 5-10° C. The reaction mixture was stirred at 5-10° C. for 3 hr. The reaction was monitored using gas chromatography. The reaction mixture was filtered and cake washed with dichloromethane (2×1 L). The filtrate was washed with 10% sodium metabisulphite (5 L), 10% sodium carbonate (10 L), dried over sodium sulphate and concentrated to give 1-ethoxycarbonyl-3-cyclopenteneoxide (19). Yield: 1.1 Kg, 98.74%.

### Example 3

### Preparation of β-ethoxycarbonylglutaraldehyde (7)

A suspension of periodic acid (1.5 Kg, 6.58 mole) in ethyl acetate (3 L) was stirred at 0-10° C. under nitrogen atmosphere. Then was added 1-ethoxycarbonyl-3-cyclopenteneoxide (19) (1 Kg, 6.40 mole) in ethyl acetate (3 L) in a drop wise manner at 0-10° C. for 1 hr. The reaction mixture was stirred at 0-10° C. for 4 hr. The reaction mixture was filtered through celite. The filtrate was washed with water (2×750 mL). The ethyl acetate layer was diluted with water (3 L). From this mixture ethyl acetate was evaporated at 30-35° C. under vacuum and aqueous layer that remained contained β-ethoxycarbonylglutaraldehyde (7). This aqueous solution was directly used in the next step.

### Example 4

### Preparation of 7-ethoxycarbonyl-9-(ethoxycarbonylmethyl)-9-azabicyclo-[3.3.1]nonan-3-one (8)

A suspension of potassium hydrogen phthalate (2.5 Kg, 12.24 mole) in water (2 L) was stirred at room temperature. Then acetonedicarboxylic acid (1.15 Kg, 8.23 mole) in water (1.4 L) and glycine ethyl ester (1.15 Kg, 8.23 mole) in water (1.6 L) were added to the reaction mixture at 15° C. to 20° C. The aqueous solution containing 13-ethoxycarbonyl glutaraldehyde (7) was added in a drop wise manner for 1 hr under nitrogen atmosphere. The reaction mixture was stirred for 12 hr at room temperature and the pH was adjusted to 8-8.5 by the addition of the potassium carbonate and extracted with ethyl acetate (3×1000 mL). The ethyl acetate layer was separated, washed with water and concentrated to give 7-ethoxycarbonyl-9-(ethoxycarbonylmethyl)-9-azabicyclo-[3.3.1]nonan-3-one (8). Yield: 1.05 Kg, 55.14%.

### Example 5

### Preparation of 7-ethoxycarbonyl-9-(ethoxycarbonylmethyl)-9-azabicyclo-[3.3.1]nonan-3-ol (9)

To a solution of 7-ethoxycarbonyl-9-(ethoxycarbonylmethyl)-9-azabicyclo-[3.3.1]nonan-3-one (8) (450 g, 1.51 mole) in ethanol (4.5 L) was added, sodiumborohydride (175 g, 4.62 mole) in a portion wise manner for 30 min at 10-15° C. The reaction mixture was stirred at room temperature for 2 hr and the pH was adjusted to 7 by the addition of the acetic acid. The solid was filtered and the filtrate was concentrated to yellow residue. Water (1.2 L) was added to the residue and the reaction mixture was basified using 10% potassium carbonate solution and extracted with ethyl acetate (3×600 mL). The ethyl acetate layer was separated and concentrated to give 7-ethoxycarbonyl-9-(ethoxycarbonylmethyl)-9-azabicyclo-[3.3.1]nonan-3-ol (9). Yield: 365 g, 80.56%.

### Example 6

### Preparation of 3-tertiary-butyl dimethylsilyloxy-7-ethoxycarbonyl-9-(ethoxycarbonylmethyl)-9-azabicyclo-[3.3.1]nonan-3-ol (20)

A solution of 7-ethoxycarbonyl-9-(ethoxycarbonylmethyl)-9-azabicyclo-[3.3.1]nonan-3-ol (9) (351 g, 1.17 mole), imidazole (239 g, 3.51 mole) and t-butyldimethylsilyl chloride (265 g, 1.7 mole) in N,N-dimethylformamide (700 mL) was stirred at 10° C. for 30 min. The reaction mixture was stirred at room temperature for 2 hr, after which it was poured into water (5 L) and extracted with ethyl acetate (3×500 ml). The ethyl acetate layer was separated, washed with water (3×1000 mL) and concentrated to give 3-tertiary butyl dimethylsilyloxy 7-ethoxycarbonyl-9-(ethoxycarbonylmethyl)-9-azabicyclo-[3.3.1]nonan-3-ol (20). Yield: 480 g, 99.17%.

¹H NMR: 200 MHz, CDCl₃; the chemical shifts expressed are in δ.

0.1 (s, 6H, 2×CH₃); 0.93 (m, 15H, 5×CH₃); 4.1 to 4.26 (m, 4H, 2×CH₂); 1.27 to 3.47 (m, 13H, 5×CH₂+3×CH).

### Example 7

### Preparation of endo-hexahydro-8-(t-butyldimethylsilyloxy)-2-ethoxycarbonyl-2,6-methano-2H-quinolizin-3-(4H)-one (21)

A mixture of 3-(t-butyldimethylsilyloxy)-7-ethoxycarbonyl-9-(ethoxycarbonylmethyl)-9-azabicyclo-[3.3.1]nonan-3-ol (20) (480 g, 1.16 mole) and potassium t-butoxide (235 g, 2.09 mole) in toluene (4.5 L) was refluxed under nitrogen atmosphere for 2 hr. Acetic acid (140 mL) was added to the reaction mixture at 10-15° C. followed by water (500 mL). The reaction mixture was extracted with ethyl acetate (3.0 L), the ethyl acetate layer was separated, washed with water and concentrated to obtain endo-hexahydro-8-(t-butyldimethylsilyloxy)-2-ethoxycarbonyl-2,6-methano-2H-quinolizin-3-(4H)-one (21). Yield: 270 g, 92.15%.

¹H NMR: 200 MHz, CDCl₃; the chemical shifts expressed are in δ.

0.08 (s, 6H, 2×CH₃); 0.89 (m, 12H, 4×CH₃); 4.1 to 4.23 (m, 4H, 2×CH₂); 1.23 to 4.2 & 4.81 to 5.3 (m, 12H, 5×CH₂+2×CH).

### Example 8

### Preparation of endo-hexahydro-8-hydroxy-2,6-methano-2H-quinolizin-3-(4H)-one (11)

To the oily compound, endo-hexahydro-8-(t-butyldimethylsilyloxy)-2-ethoxycarbonyl-2,6-methano-2H-quinolizin-3-(4H)-one (21) (100 g, 0.39 mole) in water (200 mL) concentrated hydrochloric acid (50 mL) was added. The reaction mixture was refluxed for 16 hr, cooled to room temperature and basified with potassium carbonate till pH becomes 8-8.5. This solution was concentrated under reduced pressure to obtain a residue. This residue was treated with 50% methanol in dichloromethane to precipitate inorganic material. This inorganic material was separated by filtration and filtrate was concentrated to give endo-hexahydro-8-hydroxy-2,6-methano-2H-quinolizin-3-(4H)-one (11). Yield: 26 g, 36.34%.

### Example 9

### Preparation of endo-hexahydro-8-hydroxy-2,6-methano-2H-quinolizin-3-(4H)-one (11)

To the oily compound, endo-hexahydro-8-(t-butyldimethylsilyloxy)-2-ethoxycarbonyl-2,6-methano-2H-quinolizin-3-(4H)-one (21) (100 g, 0.39 mole) in water (200 mL) was added concentrated hydrochloric acid (50 mL). The reaction mixture was refluxed for 16 h cooled to room temperature and basified with potassium carbonate till pH becomes 8-8.5. This solution was extracted with n-butanol. The n-butanol layer was separated and concentrated under reduced to give endo-hexahydro-8-hydroxy-2,6-methano-2H-quinolizin-3-(4H)-one (11). Yield: 25.5 g, 35.64%.

### Example 10

### Preparation of endo-hexahydro-8-hydroxy-2,6-methano-2H-quinolizin-3-(4H)-one (11)

To the oily compound endo-hexahydro-8-(t-butyldimethylsilyloxy)-2-ethoxycarbonyl-2,6-methano-2H-quinolizin-3-(4H)-one (21) (100 g, 0.39 mole) in water (200 mL) was added concentrated hydrochloric acid (50 mL). The reaction mixture was refluxed for 16 hr and cooled to room temperature and basified with potassium carbonate till pH becomes 8-8.5. This solution was saturated with sodium chloride and extracted with isopropanol. The isopropanol layer was separated and concentrated under reduced pressure to give residue. This residue was treated with dichloromethane and clear solution of dichloromethane was filtered and concentrated to provide endo-hexahydro-8-hydroxy-2,6-methano-2H-quinolizin-3-(4H)-one (11). Yield: 25.5 g, 35.64%.

### Example 11

### Preparation of endo-hexahydro-8-(3-indolylcarbonyloxy)-2,6-methano-2H-quinolizin-3(4H)-one (Dolasetron Base)

A solution of trifluoroacetic anhydride (413.7 g, 1.97 mole) in dichloromethane (1700 mL) was stirred under nitrogen atmosphere and to this, indole-3-carboxylic acid (302 g, 1.87 moles) was added in a portion wise manner for 30 min at −5° C. to 0° C. The reaction mixture was stirred further 30 min at −5° C. to 0° C. Then endo-hexahydro-8-hydroxy-2,6-methano-2H-quinolizin-3-(4H)-one (170 g, 0.939 moles) in dichloromethane (850 mL) was added in a drop wise manner for 30 min at −5° C. to 0° C. and was added dimethyl amino pyridine (1.43 g). The reaction mixture was stirred further for 12 h at room temperature. The reaction mixture was filtered and the collected solid washed with dichloromethane (3×170 mL). The solid was stirred in water (2550 ml) and 10% sodium carbonate (1360 mL) for 30 min. The solid formed was filtered and washed with water. This solid was stirred with 5% methanesulphonic acid (850 mL) for 1 h and filtered to remove excess undissolved indole-3-carboxylic acid. The filtrate was extracted with ethyl acetate (3×340 ml) and the ethyl acetate layer was separated. The aqueous acidic layer was basified with 10% sodium carbonate (850 mL), solid was separated, filtered and washed with water. The wet solid was dried (Dolasetron base).

Yield: 127 g, 42%.

### Example 12

### Preparation of endo-hexahydro-8-(3-indolylcarbonyloxy)-2,6-methano-2H-quinolizin-3(4H)-one (Dolasetron Base)

A solution of trifluoroacetic anhydride (121.8 g, 0.57 mole) in dichloromethane (750 mL) was stirred under nitrogen atmosphere and to this, indole-3-carboxylic acid (88 g, 0.54 mole) was added in a portion wise manner for 30 min at 0° C. to 5° C. The reaction mixture was stirred for further 30 min at 0° C. to 5° C. Then endo-hexahydro-8-hydroxy-2,6-methano-2H-quinolizin-3-(4H)-one (11) (50 g, 0.27 mole) in dichloromethane (500 mL) and dimethyl amino pyridine (0.42 g, 0.0039 mole) were added in a drop wise manner for 30 min at 0° C. to 5° C. The reaction mixture was stirred further for 12 h at room temperature. The reaction mixture was filtered and the collected solid washed with dichloromethane (100 mL). The solid was stirred in ethyl acetate (550 mL) and 10% sodium carbonate (500 mL) was further added. The ethyl acetate layer was separated, washed with water and concentrated to obtain crude Dolasetron base (60 g). The crude base was recrystallized from ethyl acetate-hexane to give pure Dolasetron base.

Yield: 50 g, 50.63%.

### Example 13

### Preparation of endo-hexahydro-8-(3-indolylcarbonyloxy)-2,6-methano-2H-quinolizin-3(4H)-one mesylate

Dolasetron base (50 g, 0.15 mole) was dissolved in acetone (1000 mL) and methane sulphonic acid was added (10.70 mL) drop wise over a period of 30 min at 20° C. The reaction mixture was stirred further for 2 hr. The solid formed was filtered, washed with cold acetone (50 mL) and dried. Yield (crude) 59 g, 90.77%.

### Example 14

### Preparation of endo-hexahydro-8-(3-indolylcarbonyloxy)-2,6-methano-2H-quinolizin-3(4H)-one mesylate

Dolasetron base (119 g, 0.368 moles) was dissolved in acetone (2023 mL) and treated with activated charcoal (12 g). Filtered the mixture through hyflow and the clear solution was treated with water (24 mL) and methane sulphonic acid (38.96 g, 0.405 moles) at 25° C. to 30° C. The reaction mass was stirred further for 2 hr at 0° C. to 5° C. The solid formed was filtered, washed with acetone (3×120 mL) and dried. Yield (crude) 140 g, 87%.

### Example 15

### Purification of endo-hexahydro-8-(3-indolylcarbonyloxy)-2,6-methano-2H-quinolizin-3(4H)-one mesylate, Dolasetron Mesylate

Dolasetron mesylate (140 g) was taken in water (900 ml) and extracted with ethyl acetate (3×280 ml). The aqueous layer was separated, basified with 10% sodium carbonate (320 mL). The solid obtained was filtered, washed with water and dried. This solid was dissolved in acetone (2×100 mL) and treated with activated charcoal (12 g). Filtered the mixture through hyflow and clear solution was treated with water (20 mL) and methane sulphonic acid (32.72 g, 0.341 moles) at 25° C. to 30° C. The reaction mass was stirred further for 2 hr at 0° C. to 5° C. The solid formed was filtered, washed with acetone (3×100 mL) and dried. Yield 130 g, 93%. Purity: 99.9% (HPLC).

### Preparation of Dolasetron Base Form I

### Example 16

5 g of Dolasetron base was dissolved in 5 mL of acetone at 40° C. to 50° C. The hot solution was filtered and was allowed to cool to 0° C. to 5° C. and stirred for 2 hr. The separated solid was isolated by filtration and dried at 60° C. to get Dolasetron base Form I.

### Example 17

5 g of Dolasetron base was dissolved in 5 mL of acetonitrile at 70° C. to 80° C. The hot solution was filtered and was allowed to cool to 0° C. to 5° C. and stirred for 2 hr. The separated solid was isolated by filtration and dried at 60° C. to get Dolasetron base Form I.

### Example 18

0.5 g of Dolasetron base was dissolved in 10 mL of acetone at 25° C. to 30° C. temperature. To this clear solution 30 mL hexane was added drop wise under stirring. The solution was maintained at 30° C. for 2 hr. The separated solid was isolated by filtration and dried at 60° C. to get Dolasetron base Form I.

### Example 19

0.5 g of Dolasetron base was dissolved in 20 mL of ethyl acetate at 70° C. to 80° C. temperature. The hot clear solution was added drop wise in 40 mL of hexane under stirring at maintained at 30° C. The solution was maintained at 30° C. for 2 hr under stirring. The separated solid was isolated by filtration and dried at 60° C. to get Dolasetron base Form I.

### Example 20

0.5 g of Dolasetron base was dissolved in 10 mL of THF at 25° C. to 30° C. temperature. The clear solution was added drop wise in 30 mL of hexane under stirring and maintained at 0° C. to 5° C. The resultant solution was maintained at 0° C. to 5° C. for 2 hr under stirring. The separated solid was isolated by filtration and dried at 60° C. to get Dolasetron base Form I.

### Example 21

0.5 g of Dolasetron base was dissolved in 10 mL of THF at 25° C. to 30° C. temperature. The clear solution was added drop wise in 40 mL of DIPE under stirring and maintained at 0° C. to 5° C. The resultant solution was maintained at 0° C. to 5° C. for 2 hr under stirring. The separated solid was isolated by filtration and dried at 60° C. to get Dolasetron base Form I.

### Preparation of Dolasetron Base Form II

### Example 22

0.5 g of Dolasetron base was dissolved in 10 mL of acetone at 25° C. to 30° C. To this clear solution 30 mL of DIPE was added drop wise under stirring. The resultant solution was maintained at 30° for 2 hr. The separated solid was isolated by filtration and dried at 60° C. to get Dolasetron base Form II.

### Example 23

0.5 g of Dolasetron base was dissolved in 70 mL of DIPE at 70° C. temperature. The hot solution was allowed to cool to 25° C. to 30° C. temperature. The suspension was stirred at the same temperature for 2 hr. The separated solid was isolated by filtration and dried at 60° C. to get Dolasetron base Form II.

### Preparation of Dolasetron Base Form III

### Example 24

0.5 g of Dolasetron base was dissolved in 7 mL of DMSO at 25° C. to 30° C. The clear solution was added drop wise under stirring into 30 mL of water maintained at 0° C. to 5° C. The resultant solution was maintained at same temperature for 2 hr. The separated solid was isolated by filtration and dried at 60° C. to get Dolasetron base Form III.

### Example 25

0.5 g of Dolasetron base was dissolved in 5 mL DMF at 25° C. to 30° C. The clear solution was added drop wise under stirring into 30 mL of water maintained at 0° C. to 5° C. The resultant solution was maintained at same temperature for 2 hr. The separated solid was isolated by filtration and dried at 60° C. to get Dolasetron base Form III.

### Example 26

0.5 g of Dolasetron base was dissolved in 7 mL of DMA at 25° C. to 30° C. The clear solution was added drop wise under stirring into 30 mL of water maintained at 0° C. to 5° C. The resultant solution was maintained at same temperature for 2 hr. The separated solid was isolated by filtration and dried at 60° C. to get Dolasetron base Form III.

### Example 27

0.5 g of Dolasetron base was dissolved in 15 mL of ethanol at 25° C. to 30° C. The clear solution was added drop wise under stirring into 30 mL of water maintained at 0° C. to 5° C. The resultant solution was maintained at same temperature for 2 hr. The separated solid was isolated by filtration and dried at 60° C. to get Dolasetron base Form III.

### Example 28

0.5 g of Dolasetron base was dissolved in 10 mL of THF at 25° C. to 30° C. The clear solution was added drop wise under stirring into 30 mL of water maintained at 0° C. to 5° C. The resultant solution was maintained at same temperature for 2 hr. The separated solid was isolated by filtration and dried at 60° C. to get Dolasetron base Form III.

### Example 29

0.5 g of Dolasetron base was dissolved in 5 mL of 1,4-dioxane at 100° C. temperature. The hot solution was allowed to cool to 25° C. to 30° C. temperature. The suspension was stirred at the same temperature for 2 hr. The separated solid was isolated by filtration and dried at 60° C. to get Dolasetron base Form III.

### Example 30

0.5 g of Dolasetron base was dissolved in 15 mL of ethyl acetate 80° C. temperature. The hot solution was allowed to cool to 25° C. to 30° C. temperature. The suspension was stirred at the same temperature for 2 hr. The separated solid was isolated by filtration and dried at 60° C. to get Dolasetron base Form III.

### Example 31

0.5 g of Dolasetron base was dissolved in 5 mL of isopropyl alcohol at 80° C. temperature. The hot solution was allowed to cool to 25° C. to 30° C. temperature. The suspension was stirred at the same temperature for 2 hr. The separated solid was isolated by filtration and dried at 60° C. to get Dolasetron base Form III.

### Example 32

0.5 g of Dolasetron base was dissolved in 5 mL of n-propanol at 80° C. temperature. The hot solution was allowed to cool to 25° C. to 30° C. temperature. The suspension was stirred at the same temperature for 2 hr. The separated solid was isolated by filtration and dried at 60° C. to get Dolasetron base Form III.

### Preparation of Dolasetron Base Form IV

### Example 33

0.5 g of Dolasetron base was dissolved in 30 mL of toluene at 110° C. temperature. The hot solution was filtered and allowed to cool to room temperature. The suspension was stirred at the same temperature for 8 hr. The separated solid was isolated by filtration and dried at 60° C. to get Dolasetron base Form IV.

### Example 34

0.5 g of Dolasetron base was dissolved in 15 mL of MDC at reflux temperature. The hot solution was filtered and was allowed to cool to room temperature and stirred for 2 hr. The separated solid was isolated by filtration and dried at 60° C. to get Dolasetron base Form IV.

### Example 35

0.5 g of Dolasetron base was dissolved in 10 mL of methanol at reflux temperature. The hot solution was filtered and was allowed to cool to room temperature and stirred for 2 hr. The separated solid was isolated by filtration and dried at 60° C. to get Dolasetron base Form IV.

### Preparation of Dolasetron Base Form V

### Example 36

0.5 g of Dolasetron base was taken in a clean and dry 100 mL round bottom flask fitted with air condenser. The flask was heated to 180° C. to 190° C. in an oil bath for 30 min. Then the flask was cooled to room temperature to obtain solid as Dolasetron base Form V.

What is claimed is:

1. A process for the preparation of endo-hexahydro-8-(3-indolylcarbonyloxy)-2,6-methano-2H-quinolizin-3(4H)-one or Dolasetron base having structural formula (A), 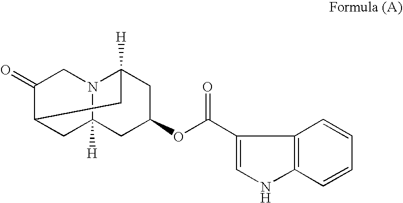
comprising:
a) reacting a compound having structural formula (4) 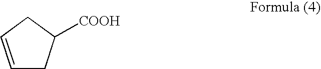
with thionyl chloride or hydrochloric acid in an alcohol to obtain a compound having structural formula (V); 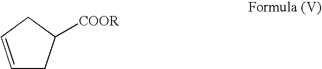
b) treating the compound (V) with m-chloroperbenzoic acid in a solvent to form an epoxide compound having structural formula (XIX); 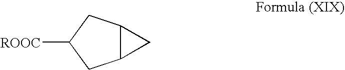
c) treating the compound (XIX) with periodic acid to obtain a compound having structural formula (VII); 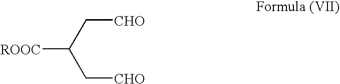
d) cyclising the compound (VII) using potassium hydrogen phthalate, acetonedicarboxylic acid and glycine ester hydrochloride in water to obtain a pseudopelletierine derivative having structural formula (VIII); 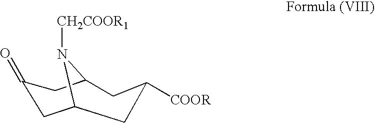
e) reducing the compound (VIII) with sodiumborohydride in an alcohol and treating it with an organic acid to obtain a compound having structural formula (IX); 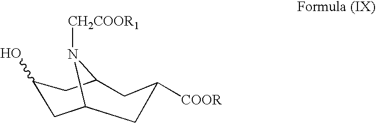
f) treating the compound (IX) with a silyl reagent, in an organic solvent to form a silyl derivative having structural formula (XX), wherein Z is a silyl group; 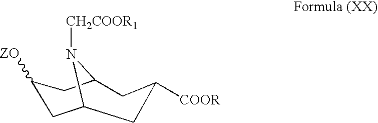
g) treating the compound (XX) with a strong base in toluene and further treating it with a mixture of organic acid and organic solvent to form a compound having structural formula (XXI); 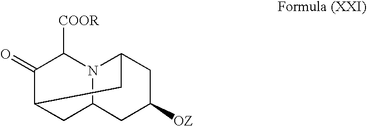
h) treating the compound (XXI) with an inorganic acid in water and treating it with an organic solvent to give a compound having structural formula (II); and 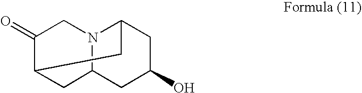
i) reacting the compound (II) with indole-3-carboxylic acid in presence of trifluoroacetic acid anhydride to obtain Dolasetron base of structural formula (A).

2. The process as claimed in claim 1, wherein R and R₁ are independently selected from a group consisting of Et, Me and OCH₂Ph.

3. The process as claimed in claim 1(a), wherein the alcohol is either methanol or ethanol.

4. The process as claimed in claim 1(b), wherein the solvent is either dichloromethane or toluene or ethyl acetate.

5. The process as claimed in claim 1(e), wherein the alcohol is either methanol or ethanol or mixture thereof.

6. The process as claimed in claim 1(e), wherein, the organic acid is selected from formic acid, methane sulphonic acid and acetic acid, or mixture thereof.

7. The process as claimed in claim 1 (e), wherein the organic acid is acetic acid.

8. The process as claimed in claim 1(f), wherein the silyl group is selected from trimethyl silyl, isopropyl dimethyl silyl, t-butyldimethyl silyl, t-butyldiphenyl silyl, tribenzyl silyl, and triisopropyl silyl.

9. The process as claimed in claim 1(f), wherein the silyl reagent is selected from trimethyl silyl chloride, isopropyl dimethyl silyl chloride, t-butyldimethyl silyl chloride, t-butyldiphenyl silyl chloride, tribenzyl silyl chloride, and triisopropyl silyl chloride.

10. The process as claimed in claim 1(f), wherein the organic solvent is selected from ketones, esters, ethers and halogenated solvents, or mixture thereof.

11. The process as claimed in claim 1(f), wherein the organic solvent is selected from acetone, tetrahydrofuran, 1,4-dioxane, dichloromethane, chloroform, N,N-dimethyl formamide, ethyl acetate, and acetonitrile.

12. The process as claimed in claim 1(g), wherein the strong base is selected from metal alkoxide.

13. The process as claimed in claim 1 (g), wherein the strong base is selected from sodium tertiary butoxide and potassium tertiary butoxide.

14. The process as claimed in claim 1 (g), wherein the organic acid is either formic acid or acetic acid or mixture thereof.

15. The process as claimed in claim 1(g), wherein the organic acid is acetic acid.

16. The process as claimed in claim 1(g), wherein the organic solvent is selected from halogenated solvents, ethers and esters, or mixture thereof.

17. The process as claimed in claim 1(g), wherein the organic solvent is selected from methylene chloride, chloroform, ethyl acetate, isopropyl acetate, diethyl ether, and diisopropyl ether, or mixture thereof.

18. The process as claimed in claim 1 (g), wherein the organic solvent is ethyl acetate.

19. The process as claimed in claim 1(h), wherein the inorganic acid is hydrochloric acid.

20. The process as claimed in claim 1(h), wherein the organic solvent is selected from alcohols, ketones and halogenated solvents, or mixture thereof.

21. The process as claimed in claim 20, wherein the organic solvent is selected from methanol, ethanol, isopropanol, n-butanol, acetone, methyl ethyl ketone, methyl isobutyl ketone, dichloromethane and chloroform, or mixture thereof.

22. The process as claimed in claim 20, wherein the organic solvent is a mixture of dichloromethane and methanol.

23. The process as claimed in claim 1(h), wherein the organic solvent is isopropanol.

24. The process as claimed in claim 1(i), wherein the ratio of indole-3-carboxylic acid and trifluoro acetic anhydride is in the range of 1:1.1 to 1:2.0

25. A process for the preparation of endo-hexahydro-8-(3-indolylcarbonyloxy)-2,6-methano-2H-quinolizin-3(4H)-one methanesulfonate (Dolasetron mesylate) having structural formula (I), comprising: 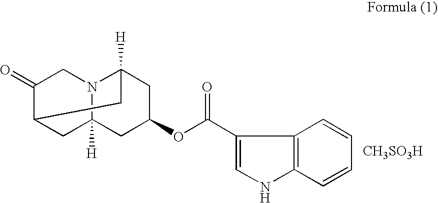
a) converting the Dolasetron base of claim 1 into its mesylate salt by treating with methane sulphonic acid in a suitable organic solvent; and
b) purifying the Dolasetron mesylate by treating with a base and further adding methanesulphonic acid to obtain highly pure compound of formula (1).

26. The process as claimed in claim 25(a), wherein the organic solvent is selected from alcohols, halogenated solvents and ketones, or mixture thereof.

27. The process as claimed in claim 26, wherein the organic solvent is selected from methanol, ethanol, isopropanol, dichloromethane, chloroform, acetone and methyl ethyl ketone, or mixture thereof.

28. The process as claimed in claim 25(a), wherein the organic solvent is acetone.

29. The process as claimed in claim 25(b), wherein the base is selected from sodium carbonate, sodium hydroxide, potassium hydroxide, and potassium carbonate.

30. The process as claimed in claim 25(b), wherein the base is sodium carbonate.

31. The process as claimed in claim 25, wherein the Dolasetron mesylate is obtained in a purity of about 99.9%.

32. A process for producing polymorphic Form I of endo-hexahydro-8-(3-indolylcarbonyloxy)-2,6-methano-2H-quinolizin-3(4H)-one (Dolasetron base), comprising: dissolving Dolasetron base in a solubilizing solvent at a temperature in the range of about 25° C. to 90° C. and adding anti-solvent to precipitate into a solid.

33. The process as claimed in claim 32, wherein the solubilizing solvent is selected from aliphatic ketones, aliphatic esters and cyclic ethers, or mixture thereof.

34. The process as claimed in claim 32, wherein the solubilizing solvent is selected from acetone, ethyl acetate, tetrahydrofuran and 1,4-dioxane, or mixture thereof.

35. The process as claimed in claim 32, wherein the anti-solvent is selected from aliphatic ethers and aliphatic hydrocarbons, or mixture thereof, provided aliphatic ketone is not used in combination with aliphatic ether.

36. The process as claimed in claim 32, wherein the anti-solvent is selected from diethyl ether, diisopropyl ether, n-hexane and n-heptane, or mixture thereof, provided acetone is not used in combination with diisopropyl ether.

37. A process for producing polymorphic Form I of endo-hexahydro-8-(3-indolylcarbonyloxy)-2,6-methano-2H-quinolizin-3 (4H)-one (Dolasetron base), comprising: dissolving Dolasetron base in a solubilizing solvent at a temperature in the range of about 25° C. to 90° C. and cooling the resultant solution at a temperature range of about 0° C. to 20° C.

38. The process as claimed in claim 37, wherein the solubilizing solvent is selected from acetone and acetonitrile.

39. A crystalline polymorphic Form II of endo-hexahydro-8-(3-indolylcarbonyloxy)-2,6-methano-2H-quinolizin-3(4H)-one (Dolasetron base) characterized by the X-ray powder diffraction pattern and FT-IR absorption spectra as given below:
Peaks in the powder X-ray diffraction pattern are at about (2θ): 7.7173, 11.2544, 11.7856, 13.0339, 13.7779, 14.2935, 15.3082, 15.6402, 15.8543, 16.3480, 16.7105, 17.3837, 18.8466, 19.0536, 21.2742, 22.1380, 22.9472, 24.5359, 24.9955, 26.2573, 26.9912, 27.7711, 28.2029, 28.6513, 29.6927, 30.5589, 31.9263, 32.7418, 33.1131, 33.9014±0.2 degrees.
Wave numbers of infrared absorption spectra are at about (cm⁻¹): 3280, 1716, 1685, 1523, 1433, 1307, 1238, 1180, 1068, 1029, 779, 754, and 717.

40. A process for producing the polymorphic Form II of Dolasetron base of claim 39 comprising: dissolving Dolasetron base in diisopropyl ether at a temperature ranging between about 60° C. and 80° C. and cooling the resultant solution at a temperature range of about −5° C. to 20° C.

41. A process for producing the polymorphic Form II of Dolasetron base of claim 39 comprising: dissolving Dolasetron base in acetone at a temperature ranging between about 20° C. and 40° C. and adding diisopropyl ether.

42. A crystalline polymorphic Form III of endo-hexahydro-8-(3-indolylcarbonyloxy)-2,6-methano-2H-quinolizin-3(4H)-one (Dolasetron base) characterized by the X-ray powder diffraction pattern and FT-IR absorption spectra as given below:
Peaks in the powder X-ray diffraction pattern are at about (2θ): 9.2394, 9.4668, 9.8411, 10.8592, 13.4933, 14.2878, 16.1274, 16.5113, 17.5210, 18.4973, 19.3556, 21.3917, 22.9458, 24.6119, 25.5599, 27.1031, 27.9029, 29.6730, 31.6704, 32.4906, 3.0179, 33.3242, 34.7579, 37.3862, 39.9142, 43.5379, 47.2834±0.2 degrees.
Wave numbers of infrared absorption spectra are at about (cm⁻¹): 3490, 1726, 1687, 1504, 1448, 1375, 1309, 1182, 1143, 1066, 1029, 798, 765, and 740.

43. A process of producing polymorphic Form III of Dolasetron base of claim 42 comprising: dissolving Dolasetron base in a solubilizing solvent at a temperature ranging from about 25° C. to 30° C., adding the solution to an anti-solvent and cooling the resulting solution.

44. The process as claimed in claim 43, wherein the solubilizing solvent is selected from polar aprotic solvents, aliphatic alcohols and cyclic ethers, or mixture thereof

45. The process as claimed in claim 43, wherein the solubilizing solvent is selected from dimethyl sulfoxide, dimethyl formamide, dimethyl acetamide, methanol, ethanol, tetrahydrofuran and 1,4-dioxane, or mixture thereof.

46. The process as claimed in claim 43, wherein the anti-solvent is water.

47. A process for producing polymorph III of Dolasetron base of claim 42 comprising: dissolving Dolasetron base in a solubilizing solvent at a temperature in the range of about 70° C. to 110° C. and cooling the solution.

48. The process as claimed in claim 47, wherein the solubilizing solvent is selected from cyclic ethers, aliphatic esters and aliphatic alcohols, or mixture thereof.

49. The process as claimed in claim 47, wherein the solubilizing solvent is selected from tetrahydrofuran, 1,4-dioxane, ethyl acetate, n-propanol and isopropanol, or mixture thereof.

50. A crystalline polymorphic Form IV of endo-hexahydro-8-(3-indolylcarbonyloxy)-2,6-methano-2H-quinolizin-3(4H)-one (Dolasetron base) characterized by the X-ray powder diffraction pattern and FT-IR absorption spectra as given below:
Peaks in the powder X-ray diffraction pattern are at about 2θ: 9.3702, 10.4465, 10.7610, 11.3038, 12.8438, 13.6345, 14.9376, 15.5688, 16.4557, 17.9327, 18.8471, 20.9858, 23.4505, 28.4316, 29.1900, 31.7488, 33.2080, 34.0855, 38.0021, 39.9206, 42.2787±0.2 degrees.
Wave numbers (cm⁻¹) of infrared absorption spectra are: 3498, 1726, 1687, 1504, 1450, 1377, 1309, 1265, 1240, 1180, 1145, 1105, 1085, 1066, 1031, 912, 798, 767, and 736.

51. A process for producing polymorph IV of Dolasetron base of claim 50 comprising: dissolving Dolasetron base in a solubilizing solvent at a temperature in the range of about 40° C. to 110° C. and cooling the solution.

52. The process as claimed in claim 51, wherein the solubilizing solvent is selected from aromatic hydrocarbon, chlorinated hydrocarbon and C₁-C₂ alcohols, or mixture thereof.

53. The process as claimed in claim 51, wherein the solubilizing solvent is selected from toluene, chloroform, methylene dichloride and methanol, or mixture thereof.

54. A crystalline polymorphic Form V of endo-hexahydro-8-(3-indolylcarbonyloxy)-2,6-methano-2H-quinolizin-3(4H)-one (Dolasetron base) characterized by the X-ray powder diffraction pattern and FT-IR absorption spectra as given below:
Peaks in the powder X-ray diffraction pattern are at about 2θ: 7.7366, 8.3350, 11.4893, 11.8951, 12.2380, 12.9631, 3.1931, 13.5550, 13.8111, 14.0013, 14.3550, 15.3181, 16.5041, 16.8688, 17.3018, 17.9151, 18.2586, 18.3637, 18.9766, 19.9918, 21.4003, 21.8024, 23.0034, 23.7376, 24.3525, 25.3915, 26.8348, 27.5637, 28.0815, 28.6278, 30.2819, 31.8967±0.2 degrees.
Wave numbers (cm¹) of infrared absorption spectra are: 1735, 1678, 1585, 1527, 1454, 1353, 1311, 1180, 1110, 1068, 1026, 912, 798, 769, 752, and 715.

55. A process for producing polymorphic Form V of Dolasetron base of claim 54 comprising: heating Dolasetron base in a temperature range of about 150° C. to 200° C.

56. The process as claimed in claim 55, wherein Dolasetron base is heated to about 195° C.
